(19) United States
(12) Patent Application Publication
(10) Pub. No.: US 2020/0364805 A1
(43) Pub. Date: Nov. 19, 2020

(54) Reducing Overages by Adjusting Share Amounts for Receiving Members of a Peer-to-Peer Needs Sharing Organization with Fixed Periodic Share Amounts

(71) Applicant: Samaritan Ministries International, Inc., Peoria, IL (US)

(72) Inventor: Nathan Riese, Peoria, IL (US)

(73) Assignee: Samaritan Ministries International, Inc., Peoria, IL (US)

(21) Appl. No.: 16/416,173

(22) Filed: May 18, 2019

(51) Int. Cl. G06Q 50/00; G16H 40/20

(52) U.S. Cl. CPC G06Q 50/01; G16H 40/20

(57) ABSTRACT

In order to reduce overages in a peer-to-peer needs sharing community, while still providing for a receiving member's complete need, shares having a predetermined amount are allocated to a receiving member's need until the remaining need amount is less than either (1) the receiving member's share or (2) the lowest share in an available shares queue. Then the receiving member's outgoing share is reduced by the receiving member's remaining need amount, and that outgoing share is added to the available needs queue.

### CROSS REFERENCES

Not Applicable.

### REFERENCE TO CDS

Not Applicable.

### FIELD OF THE INVENTION

The present disclosure relates to generating adjusted share amounts for receiving members of a peer-to-peer health care needs sharing organization to reduce overages.

### BACKGROUND

Health care sharing is an alternative to health care insurance products. Health care sharing is a type of a mutual benefit society where members commit to sharing in a portion of the medical needs of the other members. An organization can take an active role in receiving and disbursing the funds or a more passive role by publishing share information to the members to engage in a peer-to-peer direct sharing model.

In a health care sharing model, generally, each member pays a periodic share, commonly monthly. When a member has a qualifying health care expense, the member may submit that expense as a need. The health care sharing organization allocates a share from one or more other members to meet that need. The need can be met by the other members directly, in a peer-to-peer direct sharing model, or indirectly where the members send their share money to the health care sharing organization to oversee distribution of the funds to the member in need.

Monthly share amounts may vary per membership depending on the number of individuals in the membership, the age of one or more individuals in the membership, the personal responsibility selected by the member for an initial or annual unpublishable amount, and one or more health factors of the individuals in the membership. Once determined, however, the monthly share amount is generally fixed for each sharing period.

Medical bills submitted by a receiving member can vary and rarely correspond precisely with the sum of the fixed share amounts. In order to meet the needs of the receiving member in a peer-to-peer needs sharing organization, an excess number of shares may be sent to the receiving member resulting in an overage.

### SUMMARY

In order to reduce overages due to oversharing while still providing for a receiving member's complete need, shares are allocated to a receiving member's need until the remaining need amount is less than the receiving member's share, then the receiving member's outgoing share is reduced by the receiving member's remaining need amount.

Each member of the peer-to-peer needs sharing community is assigned a share to pay to another member. The share has a fixed share amount. In order to receive assistance with a health care bill, the member submits a record to the health care sharing organization. A member submitting a bill that is publishable in an allocation period may be referred to as a receiving member. Receiving members send a share, even during the month when the receiving member's medical need is published for sharing. The need has a need amount corresponding to either the total charge or a revised amount.

The needs sharing organization prepares a needs queue and an available shares queue. An allocation engine is computer run software that matches shares with needs, as the peer-to-peer direct sharing community grows the process of allocation becomes more complex. The allocation engine allocates a share that is less than the outstanding amount of the need from the available shares queue, and associates that share with an assigned share list associated with the receiving member's need or unique member identification number of the receiving member. The allocated share is then removed from the available shares queue. As shares are allocated to the assigned list, the allocation engine generates an assigned share total being the sum of the at least one assigned share amount in the assigned share list. The allocation engine also generates a remaining need amount that is the original need amount less the assigned share total. Allocation is an iterative process, continuously assigning shares from the available shares queue until a stopping point. The stopping point can be when the remaining need amount is less than the available share amount of the smallest available share. Alternatively, the stopping point can be when the remaining need amount is less than the receiving member's outgoing share. Then the receiving member's outgoing share amount is reduced by the receiving member's remaining need amount. A first share slip is generated and delivered to the receiving member showing the adjusted share amount.

When a peer-to-peer needs sharing community utilizes a fixed share amount, then there are complications encountered. As the size of the needs sharing community expands, those complications increase. For example, the allocation process for 10,000 needs with 80,000 fixed share amounts requires an allocation engine to allocate shares efficiently. I recognized that an allocation engine can be optimized by starting with largest share and then assigning subsequent shares of decreasing amount until the remaining need amount is less then the lowest available share in the available shares queue. In order to avoid over-sharing, the receiving member's outgoing share can be reduced the difference between the total need amount and the sum of the assigned shares.

In order to accomplish this, a receiving member's share must not be allocated until after the receiving member's need has been recalculated for the adjusted share amount. The allocation engine does not know how much the receiving member will send until after that member's need has been allocated. The adjusted share amount becomes that member's outgoing share. During the allocation of a subsequent need, the available shares queue contains a new variant share amount, the first receiving member's adjusted share amount. This is advantageous because subsequent needs have a great pool of share amounts to more precisely reach the need amount.

The allocation engine may be configured to prevent reduction of an outgoing share to less than a specified amount, for example under $25. This prevents a member from sending an absurdly low outgoing share, for example the allocation engine would not reduce a $500 outgoing share by $499.99 resulting in an outgoing share of $0.01. The allocation engine is also configured to prevent a receiving member's outgoing share from being completely credited in order to ensure that all members send a share. There may be, however, certain receiving members who do not send a share, such as inactivated members. An inactivated member is a member who submitted a need during their membership, but then terminated their membership. The inactivated member may be allocated later in the order of needs allocated so that the available share queue has the most diverse population of share amounts. By having a diverse population of share amounts, the allocation engine is able to more precisely meet the non-sharing member's need without overage.

Some of the advantages of the current disclosure include:
a. enables allocation engine to meet a receiving member's need more precisely;
b. receiving member waits for one less share than without self-sharing;
c. receiving members may be responsible for tracking received shares, this reduces the responsibility to track shares;
d. the receiving member's cash flow is aided by reducing the amount that Member is required to send out in their outgoing share;
e. reduces collection efforts for receiving members who receive an overage.

### DETAILED DESCRIPTION

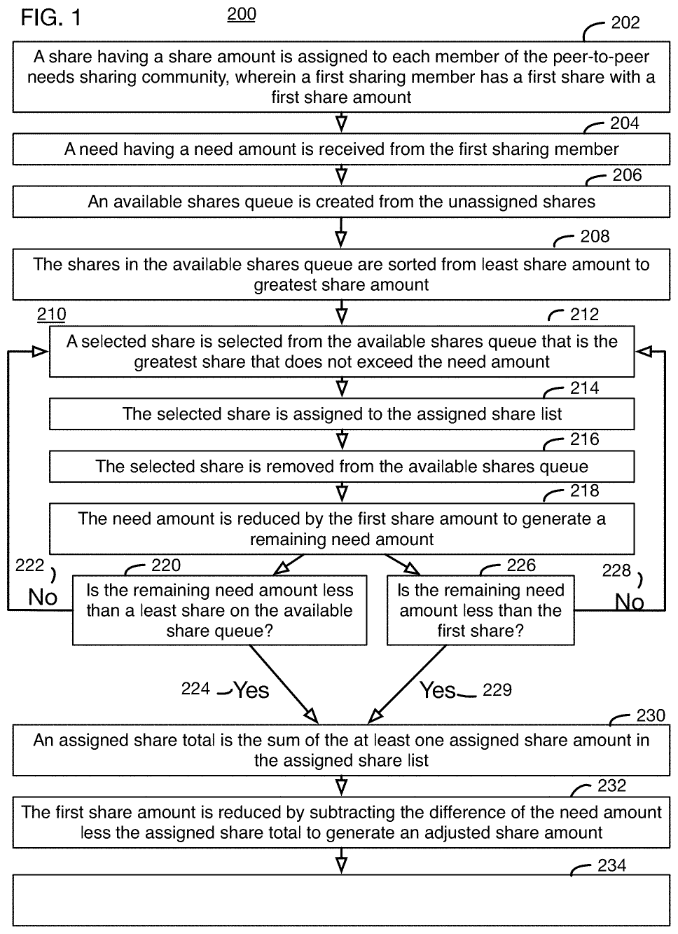

A peer-to-peer needs sharing community enables members to share in one another's needs without overages by generating share slips to receiving members showing an adjusted outgoing share amount according to the self-share reduction process 200 described in FIG. 1. Needs sharing communities are commonly set up to share the financial burden of health care costs. Memberships of the peer-to-peer needs sharing community may correspond to households of at least one person, such as a family unit. According to step 202, the dollar amount of the share amount is assigned to each member. The share amount can be determined based on the number of individuals in the household. Additionally, the dollar amount of the share amount can change based on the relationship of the individuals to one-another, the age of the individuals, the health history of the individuals in the membership, the current health status of the individuals in the membership, and the member's desire to be responsible for a portion of their own health care costs. For example, the share amount for a membership of a single person may be less expensive than a membership of a two-person member. And a two-person membership may have a share amount that is less expensive than a membership of three or more individuals. For example, the current health status of the individuals could affect the dollar amount of the share by measuring the body mass index of the individuals in the membership, with a lower dollar amount for individuals with a lower body mass index. The membership may also desire to take personal responsibility for a portion of their own health care costs, electing a higher amount of an initial unpublishable amount or an annual or per medical incident personal responsibility amount. These are amounts that the member is responsible for paying, which may incentivize the member to obtain reasonably priced health care services. Members who take greater personal responsibility for their health care cost payments may be assigned a lower share amount than those members who choose a lesser personal responsibility. The member may be able to select this share amount at sign up and may be able to change the amount at certain times during the membership term. Once a share amount is assigned to each member for a period, then the sharing organization knows the total shares available amount. The period may be monthly, weekly, or some other set period.

According to step 204, the share organization receives needs from its members during the period. When a member submits a bill to be published amount the membership, that member may be referred to as a receiving member. The receiving member may submit one or more bills, such as medical bills, which can be grouped into a need. The need has a need amount which is the sum of the bills. Receiving members send a share, even during the month when the receiving member's medical need is published for sharing. The need has a need amount corresponding to either the total charge or a revised amount.

When the need corresponds to health care, the amount that can be shared may not equal the sum of all of the bills. Medical billing is complex and may benefit from the use of medical bill negotiation service or repricing to a usual and customary amount to arrive at a publishable bill amount. Publishing is the process by which the members are notified of their share amount and assigned recipient. There are numerous companies who provide such medical bill negotiations or re-pricing for usual and customary prices. In those circumstances, the need amount corresponds to the medical bill negotiation price or the re-priced usual and customary price.

According to step 206, an available shares queue is created from the unassigned shares. The available shares queue contains a list of the share amounts associated with a unique membership identification number. Certain shares may be excluded from the available shares queue. For example, shares from receiving members may be excluded from the available shares queue until that receiving member's need has been allocated. The available shares queue may also exclude shares from inactivated members, as discussed below.

According to step 208, the shares in the available shares queue are sorted from least share amount to greatest share amount. The needs in the needs queue may also be sorted from least need amount to greatest need amount. The system may prioritize assigning the largest shares to the largest needs, in order to send the fewest number of shares to the receiving members.

The allocation engine begins an iterative process 210. The iterative process 210 starts according to step 212 by selecting a selected share from the available shares queue that is the greatest share that does not exceed the need amount.

According to step 214, the selected share is assigned to the assigned share list. The assigned share list is associated with a need identification number or a unique membership identification of the receiving member.

According to step 216, the selected share is then removed from the available shares queue. This is to prevent double allocation of a share.

According to step 218, the need amount is reduced by the first share amount to generate a remaining need amount. The allocation engine tracks the remaining need amount through each iteration as an intermediary reduced need amount. During each iteration of the iterative process 210, the allocation engine calculates the intermediary reduced need amount reduced the original need amount by each subsequent selected share. This process continues until the last iteration when the intermediary reduced need amount is determined to have reached a stopping point. The allocation engine stops the iterative process 210 upon a stopping point through either step 220 or step 226.

According to step 220, the iterative process 210 stops when the remaining need amount less than a least share on the available shares queue. According to path 222, if the remaining need amount is greater than the least share in the available shares queue, then the allocation engine returns to step 212 to select and assign a subsequent share. This iterative process of selecting and assigning subsequent shares continues through subsequent iterations. According to path 224, when the remaining need amount is less than the least share on the available shares queue, then the allocation engine proceeds to step 230.

According to step 226, the iterative process 210 stops when the remaining need amount less than the receiving member's outgoing share. According to path 228, if the remaining need amount is great than the receiving member's outgoing share, then the allocation engine returns to step 212 to select and assign a subsequent share. This iterative process of selecting and assigning subsequent shares continues through subsequent iterations. The allocation engine may also be configured to calculate a maximum share reduction in step 226, wherein the allocation engine analyzes whether the remaining need amount less than $25 less than the first share. This prevents a receiving member from sending an outgoing share of a small amount, such as an amount under $25. This amount can vary depending on the cost of mailing the share for paper checks, which has greater mailing costs associated with it then, for example, with an electronic payment. An electronic payment may have fees associated with it, however, and so the minimum payment should correspond to a reasonable amount for the receiving member to transfer in light of the transaction costs. According to path 229, when the remaining need amount is less than the receiving member's outgoing share, then the allocation engine proceeds to step 230.

According to step 230, the allocation engine determines an assigned share total which is the sum of the at least one assigned share amount in the assigned share list upon reaching a stopping point. The stopping point determines the last iteration for a selected need.

According to step 232, the receiving member's outgoing share amount is reduced. The allocation engine subtracts the assigned share total from the receiving member's need amount. The allocation engine generates a self-share adjusted share amount by reducing receiving member's outgoing share amount by the difference of the need amount less the assigned share total. This self-share adjusted share amount becomes the receiving member's outgoing share amount. The self-share adjusted share amount is then added to the available shares queue. The available shares queue may then be resorted from least share amount to greatest share amount, inclusive of the receiving member's self-share adjusted share amount.

According to step 234, a first share slip is generated for the first sharing member (also referred to as the receiving member) showing the self-share adjusted share amount. This first share slip is generated once the first sharing member's self-share adjusted share amount is selected by the allocation engine and assigned to a subsequent need. The first share slip can be delivered by paper or electronically. The share slip may indicate the receiving member's name, receiving member's mailing address, and a brief description of the need. An online share slip may be accessible through a member portal utilizing an electronic graphical user display. Members who receive online share slips may be presented with clickable web links for e-payment services. The online share slip may also include a clickable web link for the sending member to indicate their intention to send the share through paper mail or an e-service.

The iterative process 210 proceeds with the outgoing shares queue, a first predetermined share amount associated with a first member, and a first bill amount associated with the first member. The allocation engine begins to assign shares from the available shares queue to needs. In one embodiment, the system starts with the largest need ready for publication to the members for sharing during the period. The system matches the share from the available shares queue having the largest share amount that does not exceed the need amount.

The self-share reduction process 200 reduces overages due to oversharing, while still providing for a receiving member's complete need.

The allocation engine is computer run software that matches shares with needs. The allocation engine allocates a share that is less than the outstanding amount of the need from the available shares queue, and associates that share with an assigned share list associated with the receiving member's need. The allocated share is then removed from the available share queue. As shares are allocated to the assigned list, the allocation engine generates an assigned share total being the sum of the at least one assigned share amount in the assigned share list. The allocation engine also generates a remaining need amount that is the original need amount less the assigned share total.

The first share slip is then published and presented to the first sharing member. Requiring the receiving member to send a share during a receiving month encourages participation and the community aspect of sharing. The self-share adjusted share amount also provides unique share amounts for the allocation engine to allocate shares that more precisely fit the need amount.

An alternative method for precisely matching needs to shares is to split a share, so that a single member is sending a first portion of their share to a first receiving member and a second portion of their share to a second receiving member. This split share process is more burdensome on the sending member and decreases the personalization of the peer-to-peer sharing. The self-share reduction process 200 increases the number of members sharing with not more than one individual during a given period at a predetermined share amount.

The allocation engine is also configured to prevent a receiving member's outgoing share from being completely credited in order to ensure that all members send a share. There may be, however, certain receiving members who do not send a share, such as inactivated members, who may be referred to as non-sharing members. An inactivated member is a member who submitted a need during their membership, but then terminated their membership. The inactivated member may be allocated later in the order of needs allocated so that the available share queue has the most diverse population of share amounts. By having a diverse population of share amounts, the allocation engine is able to more precisely meet the non-sharing member's need without overage.

We claim:

1. A process for generating a share slip for a member of a peer-to-peer needs sharing community, the process comprising:
a. assigning a share having a share amount to each of a plurality of members of the peer-to-peer needs sharing community, wherein a first sharing member has a first share with a first share amount;
b. receiving a first need having a need amount from the first sharing member;
c. allocating at least one assigned share to an assigned share list associated with the first need from the first sharing member;
d. generating an assigned share total being a sum of the at least one assigned share in the assigned share list;
e. reducing the first share amount by the difference of the need amount less the assigned share total to generate an adjusted share amount; and
f. generating a first share slip for the first sharing member showing the adjusted share amount.

2. The process of claim 1, wherein the step of allocating at least one assigned share to an assigned share list associated with the first need from the first sharing member comprises the substeps of:
a. creating an available shares queue from a plurality of unassigned shares;
b. sorting the available shares queue from least to greatest based on the share amount of the share;
c. selecting a selected share from the available shares queue wherein the selected share is the greatest share that does not exceed the need amount;
d. assigning the selected share to the assigned share list;
e. removing the selected share from the available shares queue;
f. reducing the need amount by the first share amount to generate a remaining need amount; and
g. Iteratively assigning a subsequent share and reducing the remaining need amount by the subsequent share until the remaining need amount is less than a least share on the available shares queue.

3. The process of claim 1, wherein the step of allocating at least one assigned share to an assigned share list associated with the first need from the first sharing member comprises the substeps of:
a. creating an available shares queue from a plurality of unassigned shares;
b. sorting the available shares queue from least to greatest based on the share amount of the share;
c. selecting a selected share from the available shares queue wherein the selected share is the greatest share that does not exceed the need amount;
d. assigning the selected share to the assigned share list;
e. removing the selected share from the available shares queue;
f. reducing the need amount by the first share amount to generate a remaining need amount; and
g. Iteratively assigning a subsequent share and reducing the remaining need amount by the subsequent share until the remaining need amount is less than the first share.

4. The process of claim 3, further comprising the step of:
a. presenting the first share slip to the first sharing member to enable the first sharing member to participate in the peer-to-peer needs sharing community.

5. The process of claim 4, wherein the first need corresponds to at least one bill related to a health care service.

6. A process for generating a share slip for a first member of a peer-to-peer needs sharing community, the process comprising:
a. generating a shares queue having a plurality of shares associated with a plurality of members of the peer-to-peer needs sharing community, wherein each of the plurality of shares has a predetermined share amount;
b. generating a needs queue having a plurality of needs received from a plurality of needing members from the plurality of members, wherein each of the plurality of needs has a need amount;
c. receiving a first need from a first member, wherein the first need has a first need amount, and the first member has a first predetermined share amount;
d. allocating a plurality of selected shares to the first need, wherein the first need is reduced by a sum of the plurality of selected shares to generate a remaining need amount that is less than the greater of: (1) the first predetermined share amount or (2) a lowest share amount from the shares queue;
e. reducing the first predetermined share amount of the first member by the remaining need amount to generate a self-share adjusted share amount; and
f. generating a share slip for the first member having the self-share adjusted share amount.

7. The process of claim 6, wherein the step of allocating the plurality of selected shares to the first need comprises the substeps of:
a. creating an available shares queue from a plurality of unassigned shares, wherein each of the plurality of shares associated with the plurality of needing members is excluded from the available share queue.

8. The process of claim 7, wherein the step of allocating the plurality of selected shares to the first need further comprises the substeps of:
a. creating an available shares queue from a plurality of unassigned shares;
b. sorting the available share queue from least to greatest based on the predetermined share amount of each of the plurality of shares;
c. selecting a selected share from the available shares queue wherein the selected share is the greatest share that does not exceed the need amount;
d. assigning the selected share to an assigned share list; and
e. removing the selected share from the available shares queue.

9. The process of claim 8, further comprising the step of:
a. adding the self-share adjusted share amount to the available shares queue.

10. The process of claim 9, wherein the step of allocating the plurality of selected shares to the first need comprises the substeps of:
a. creating an available shares queue from a plurality of unassigned shares, wherein each of the plurality of shares associated with the plurality of needing members is excluded from the available share queue.

11. The process of claim 10, wherein the step of allocating the plurality of selected shares to the first need comprises the substeps of:
a. sorting the available share queue from least to greatest based on the predetermined share amount of each of the plurality of shares;
b. selecting a selected share from the available shares queue wherein the selected share is the greatest share that does not exceed the need amount;
c. assigning the selected share to an assigned share list; and
d. removing the selected share from the available shares queue.

12. The process of claim 11, further comprising the step of:
a. adding the self-share adjusted share amount to the available shares queue.

13. The process of claim 6, further comprising the steps of:
a. creating an available shares queue from a plurality of unassigned shares;
b. sorting the available share queue from least to greatest based on the predetermined share amount of each of the plurality of shares; and
c. proceeding, starting with the available shares queue, the first predetermined share amount, the need amount, and the first need amount, to (1) select a selected share from the available shares queue wherein the selected share is a greatest share that does not exceed the need amount, (2) assign the selected share to an assigned shares list for the first member, and (3) remove the selected share from the available share queue, wherein substeps 1-3 are performed in an iterative manner to generate an intermediary reduced need amount, with the intermediary reduced need amount being reduced by a subsequent selected share in a next iteration, until the intermediary reduced need amount is less than the first predetermined share amount.

14. A process for generating a share slip for a first receiving member of a peer-to-peer needs sharing community, the process comprising:
a. generating an available shares queue having a plurality of shares assigned each member of the peer-to-peer needs sharing community, wherein each share has a predetermined share amount, and the first receiving member has a first share having a first predetermined share amount;
b. receiving a first bill from the first receiving member, wherein the first bill has a first bill amount;
c. proceeding, starting with the available shares queue, the first predetermined share amount, and the first bill amount, to assign a first share from the available shares queue to the first receiving member in an iterative manner to generate an intermediary reduced need amount, with the intermediary reduced need amount being reduced by a subsequent share from the available shares queue in a next iteration, until the intermediary reduced need amount is less than the first predetermined share amount; and
d. generating a share slip for the first receiving member showing the first predetermined share amount reduced by the intermediary reduced need amount after a last iteration.

15. The process of claim 14, wherein the step of generating an available shares queue further comprises:
a. excluding the first share from the available shares queue.

16. The process of claim 15, further comprising the step of:
a. adding the first share having an amount equal to the first predetermined share amount less the intermediary reduced need amount to the available shares queue after the last iteration.

17. The process of claim 16, further comprising the step of:
a. Selecting a need from a non-sharing members later than a need from a sharing members.
